(19) United States
(12) Patent Application Publication
(10) Pub. No.: US 2002/0022795 A1
(43) Pub. Date: Feb. 21, 2002

(54) Bilayer electrodes

(76) Inventors: John R. Reynolds, Gainesville, FL (US); Hiep Ly, Webster, NY (US); Patrick John Kinlen, Fenton, MO (US); Vinod P. Menon, Woodbury, MN (US)

Correspondence Address: THOMPSON COBURN, LLP, ONE FIRSTAR PLAZA, SUITE 3500, ST LOUIS, MO, 63101, US

(21) Appl. No.: 09/929,584

(22) Filed: Aug. 14, 2001

Related U.S. Application Data
(60) Provisional application No. 60/225,193, filed on Aug. 14, 2000.

(51) Int. Cl. A61L 2/00; B05D 3/00; A61N 1/30

(52) U.S. Cl. 604/20; 427/2.1; 424/400

(57) ABSTRACT

The invention herein comprises a polymer bilayer which eliminates the spontaneous release of an active molecule by ion exchange. The spontaneous release is slowed by the application of a second polymer layer to the top of the electroactive polymer. The electroactive bilayer is preferably polypyrrole. The second polymer layer is preferably poly(vinyl butyral), poly(vinyl acetate) or nafion. This second layer stops the spontaneous release of the active molecule. The active molecule will usually be a pharmaceutical agent or drug. This bilayer allows a more effective controlled drug delivery system wherein the quantities and timing of the drugs can be given to the human being/animal patient with a high degree of certainty and predictability.

### BACKGROUND OF THE INVENTION

This invention relates to controlled drug release systems. More particularly this invention relates to controlled drug release systems having a releasable dopant therewith or thereon.

Controlled drug delivery (“CDD”) is an area of great interest in the medical community. Some of the advantages that a CDD system offers include: 1) a higher degree of control over the rate and duration of drug release, 2) localized treatment of a target area, which leads to lower dosages and fewer side effects and 3) the possibility of self-regulated drug delivery. A significant amount of research has been focused on the use of polymeric materials as controlled drug delivery systems. See Park, K., Ed.; Controlled Drug Delivery, Challenges and Strategies, ACS Press, Washington, D.C., 1997. Many of these CDD systems being developed are based on the type of stimulus which is available or that can be used to trigger release of the drug at the target site. Some of the types of systems currently being developed are pH responsive (See Okano, T., Ed.; Biorelated Polymers and Gels: Controlled Release Applications in Biomedical Engineering, Academic Press, San Diego, 1998, 103), temperature responsive (Ibid; 107) and electrochemically responsive (Ibid; 110).

Electroactive-conducting polymers provide a very promising basis for the development of electrochemically responsive CDD systems. There have been many studies aimed at using polypyrrole and/or its derivatives as the host polymer for ion transport/release. See Sundaresan, N. S.; Basak, S.; Pomerantz, M.; Reynolds, J. R.; J. Chem. Soc. Commun.; 1987, 621; Ren, X.; Pickup, P. G.; J. Phys. Chem.; 1993, 97, 5356; Pyo, M.; Maeder, G.; Kennedy, R. T.; Reynolds, J. R.; J. Electroanal. Chem.; 1994, 368, 329; Demoustier-Champagne, S.; Reynolds, J. R. Chem. Mater.; 1995, 7, 277; Komura, T.; Goisihara, S.; Ymaguti, T.; Takahasi,, K.; J. Electroanal. Chem.; 1998, 456, 121). The loading and release of ionic drugs and biomolecules into and out of these electroactive systems is achieved via ion movement into and out of the polymer matrix which helps to maintain charge neutrality as the electroactive polymer undergoes electrochemical switching. In a recent study, Schenoff et. al were able to demonstrate and study this charge compensation process using a poly(butyl violgen)/polystyrene sulfonate system. See Schenoff, J. B.; Ly, H.; Li, M.; J. Am. Chem. Soc.; 1998, 120, 7626.

However, a major lingering concern regarding a drug delivery system using electroactive polymers has been the spontaneous release of an active molecule(s) by ion exchange. Reynolds, J. R.; Ly, H.; Fatma, S. and Kinlen, P. J.; Polym. Prepr.; 40(1)307(1999).

### OBJECTS OF THE INVENTION

An object of this invention is to provide a process for slowing down or repressing the spontaneous release rate of active molecules by ion exchange in electroactive polymers containing active biomolecules.

It is another object of this invention to provide a CDD system with a higher degree of control over the rate and duration of drug release therefrom.

It is also an object of this invention to provide a CDD system for the localized treatment of a target area, with lower dosages and fewer adverse patient side effects.

It is yet another object of this invention to provide a CDD system having a high degree of control.

It is still another object of this invention to provide a process for. impeding the unwanted or undesired spontaneous exchange of biologically active molecules from an electroactive polymer containing the same while still maintaining the desired burst release characteristics of a CDD system.

The aforementioned and other objects are met in this invention which is described in more nonlimiting detail hereinafter.

### BRIEF SUMMARY OF THE INVENTION

In one embodiment, this invention comprises a controlled drug release electrode system comprising an electroactive polymer having an ionic exchangeable releasable dopant thereon and an effective conforming thickness of a water insoluble film forming overlayer substantially impermeable to said dopant.

In another embodiment, this invention comprises a process for preparing a controlled drug release electrode system comprising an electroactive polymer having an ionic exchangeable dopant thereon and additionally an effective conforming thickness of a water insoluble film forming overlayer substantially impermeable to said dopant thereon which process comprises the effective application of said film forming overlayer in an adherent fashion to said polymer.

This invention further comprises a method for treating a patient(s) using a controlled drug release electrode system comprising an electroactive polymer having an ionic exchangeable dopant thereon and an effective conforming thickness of a water insoluble film forming overlayer substantially impermeable to said dopant, which comprises contacting a patient with this electrode system and applying an effective potential to the electrode when the electrode is in contact with a patient whereby said drug is released from the polymer and is made effectively available to the patient.

Further herein described is a process of producing an electrochemical responsive controlled drug delivery system wherein a film of an electroactive polymer, loaded with an active ingredient, has a second polymer layer applied thereto, allowing said second polymer layer to dry.

### BRIEF DESCRIPTION OF THE DRAWINGS

FIG. 1 illustrates a simple model for mono-anion release from an electroactive polymer film.

FIG. 2 depicts the application of an applied potential to reduce the film leads to an immediate and rapid salicylate release.

FIG. 3 depicts spontaneous exchange conditions in the depletion of a drug reservoir.

FIG. 4 is a representation of the spontaneous release process.

FIG. 5 is a representation of the use of an overlayer in order to stop or limit the spontaneous ion exchange process.

FIG. 6 indicates that the presence of the PVB overlayer significantly reduced the amount of salicylate that is spontaneously released.

FIG. 7 shows results from an experiment in which PVB is initially present and then removed.

FIG. 8 depicts data indicating that the salicylate release from the Nafion coated PP/salicylate system exhibited release behavior similar to that of the PVB coated system.

FIG. 9 indicates that the PP/salicylate system with the 88% hydrolyzed PVA overlayer was not effective at impeding spontaneous release.

FIGS. 10 and 11 evidence that subjecting a 40% hydrolyzed PVA overlayer to similar crosslinking conditions resulted in a dramatic difference in the salicylate release characteristics.

### DETAILED DESCRIPTION OF THE DRAWINGS

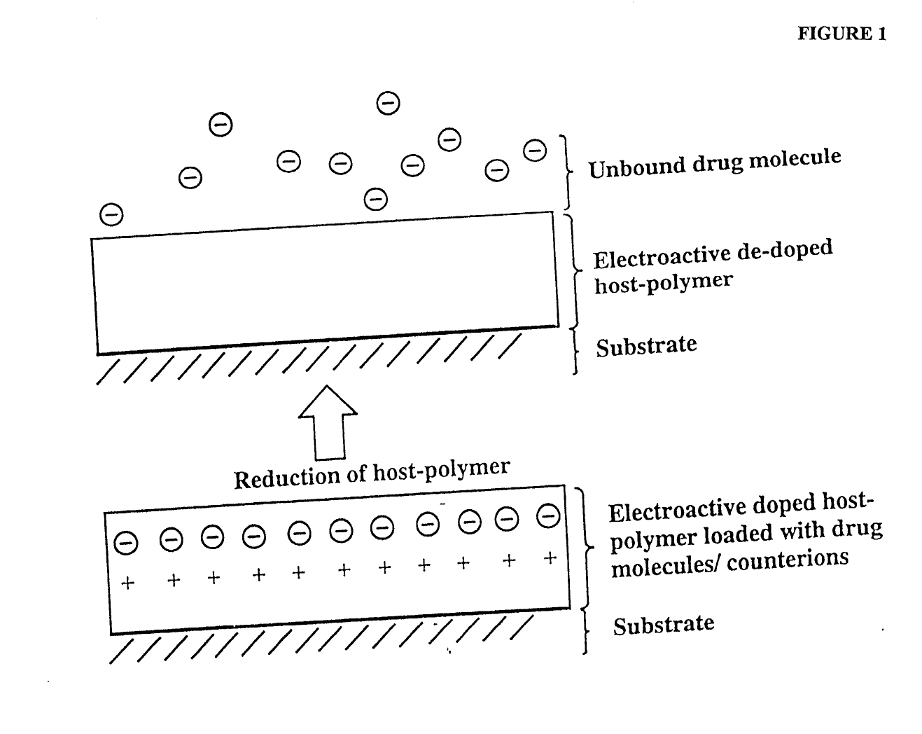

FIG. 1 illustrates a simple model for mono-anion release from an electroactive polymer film. Illustratively in carrying out this invention, the loading of the anionic drug into the polymer matrix is carried out as part of the polymerization process; whereby the monomer is electropolymerized in the presence of the salt of the anionic dopant. The anionic drug is incorporated into the polymer matrix to maintain charge neutrality of the polymer system and this ion transport into the polymer matrix is driven by electrostatic interactions between the positively charged polymer and the negatively charged dopant ions.

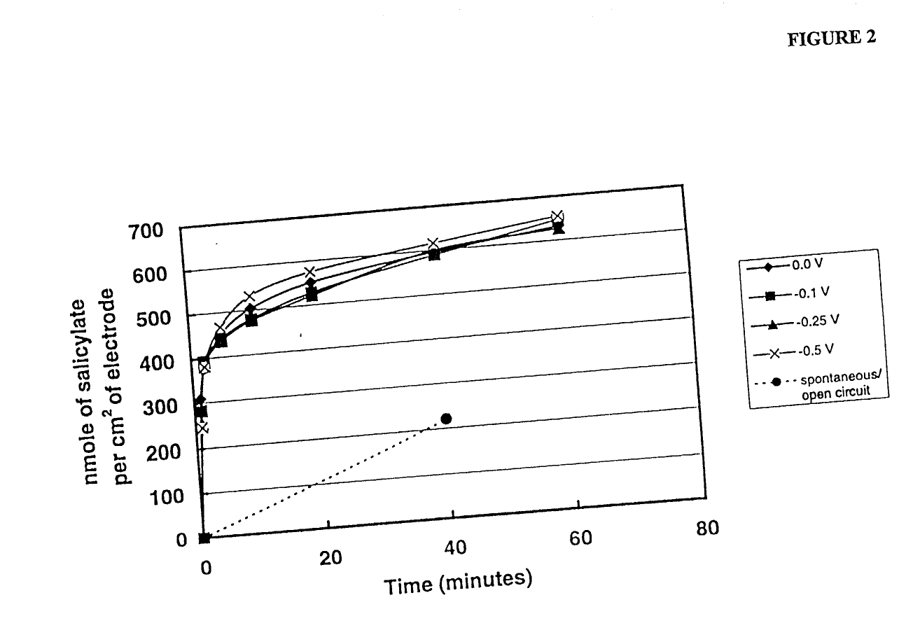

FIG. 2 depicts that the application of an applied potential to reduce the film leads to an immediate and rapid salicylate release.

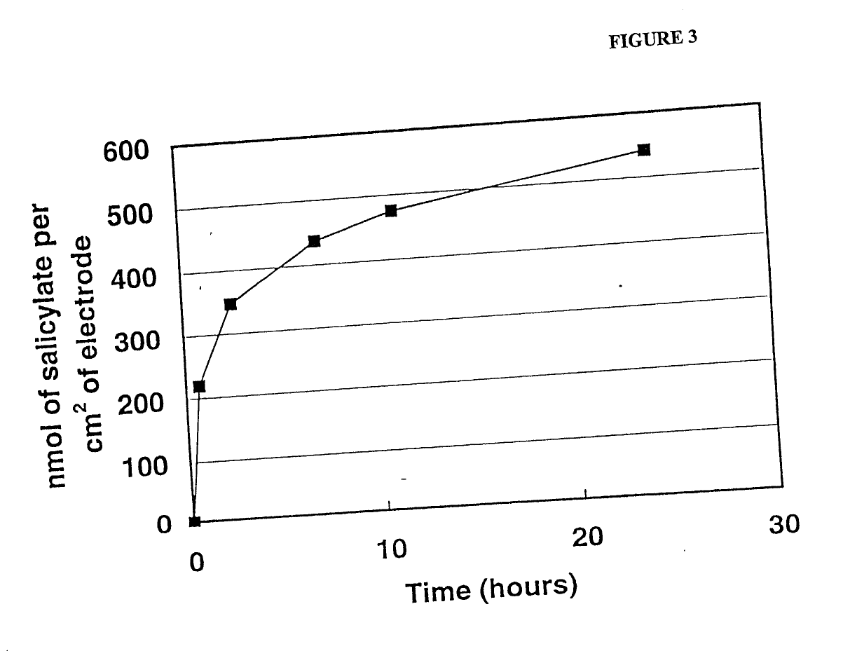

FIG. 3 indicates that under similar spontaneous exchange conditions (immersion of PP/salicylate in buffer with no applied potential), the entire drug reservoir can be depleted within a 24 hour period.

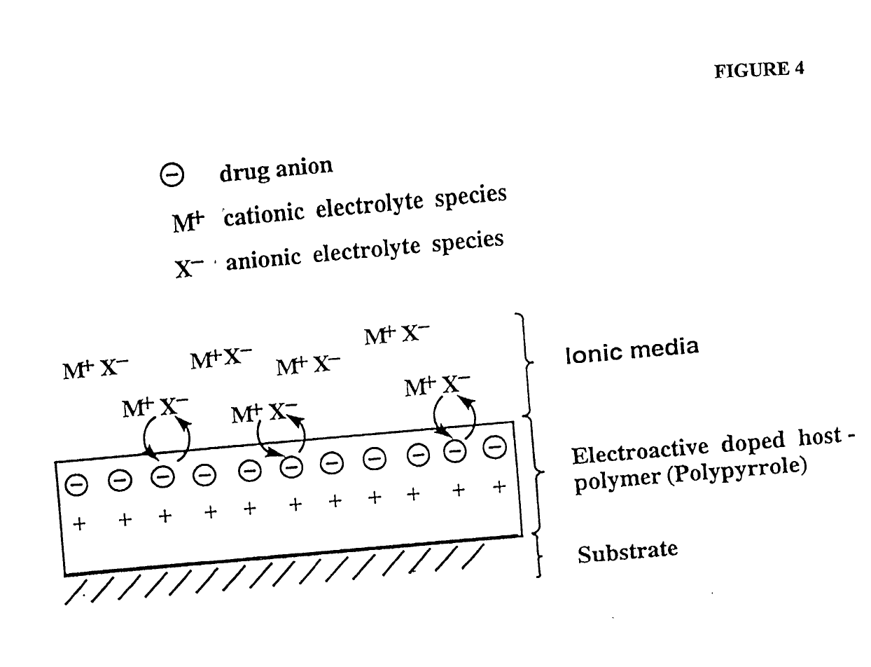

FIG. 4 is a representation of the spontaneous release process. The process in these CDD systems is believed to be a simple ion-exchange phenomenon in which drug molecules within the polymer matrix are exchanged with species of the same charge existing in the electrolyte media.

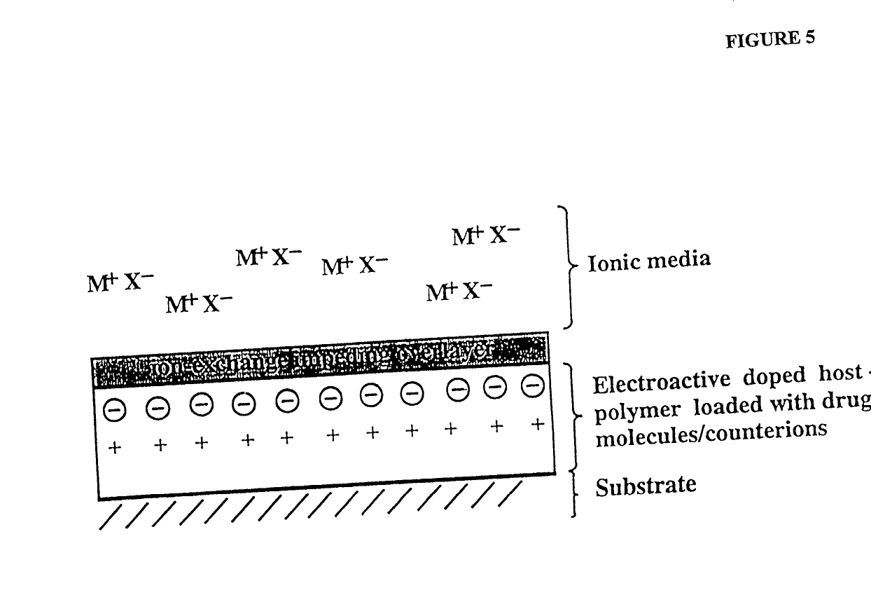

FIG. 5 is a representation of the use of an overlayer in order to stop or limit the spontaneous ion exchange process. A polymer overlayer is deposited on top of the loaded CDD system to limit the interaction between the drug molecules in the CDD system matrix and species of similar charge in the ionic media.

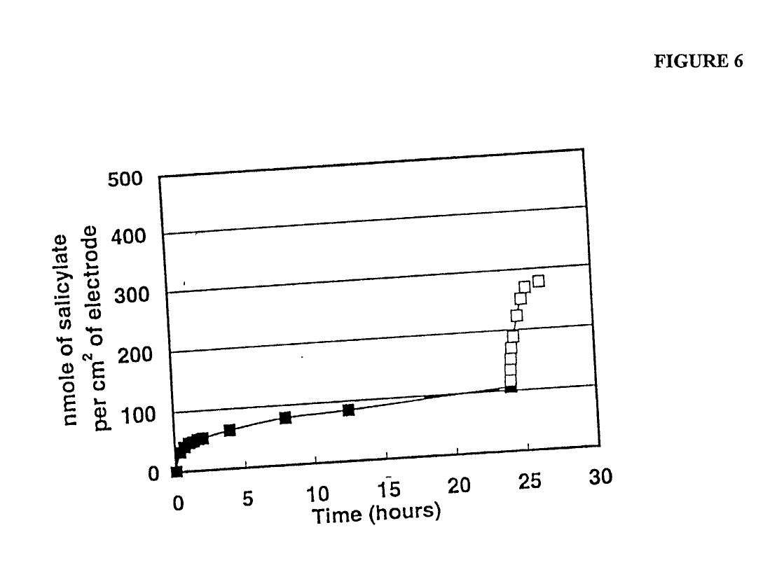

FIG. 6 indicates that the presence of the PVB overlayer significantly reduced the amount of salicylate that is spontaneously released over a 24 hour period.

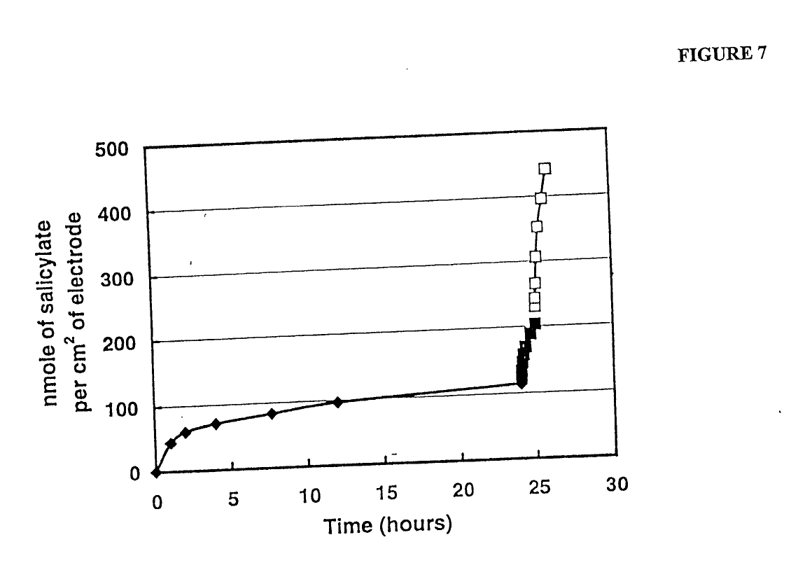

FIG. 7 shows data from an experiment in which the PVB is initially present and then removed using THF after the applied potential release begins to subside. Removal of the overlayer allowed for a higher rate of applied potential release to take place. The results indicate that the PVB overlayer not only inhibits spontaneous release, but also hinders normal applied potential release.

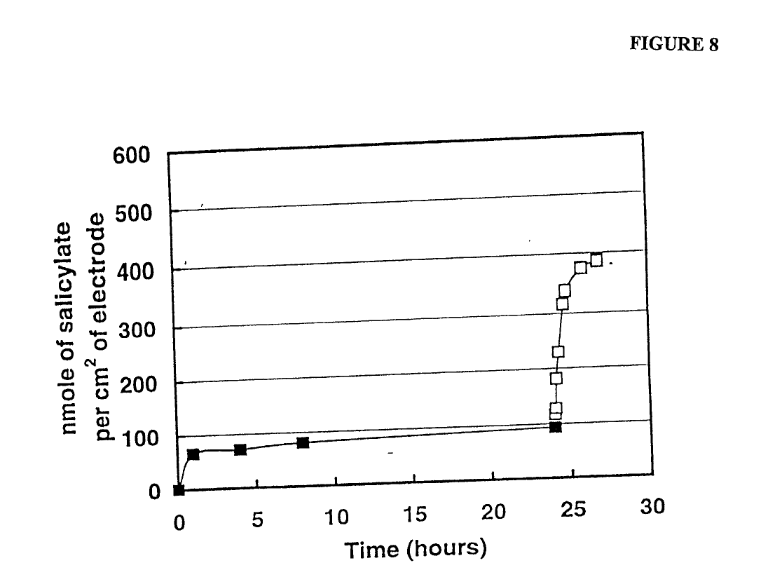

FIG. 8 indicates that the salicylate release from the Nafion coated PP/salicylate system exhibited release behavior similar to that of the PVB coated system. However, the ratio of spontaneous release to applied potential release for the Nafion overlayer is approximately 1:3; while the ratio for the PVB overlayer is approximately 1:2.

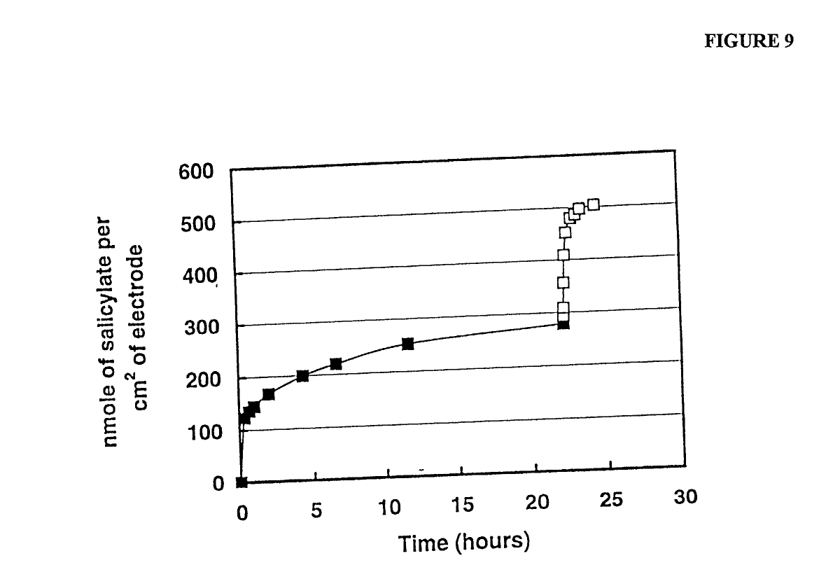

FIG. 9 indicates that the PP/salicylate system with the 88% hydrolyzed PVA overlayer was not effective at impeding spontaneous release.

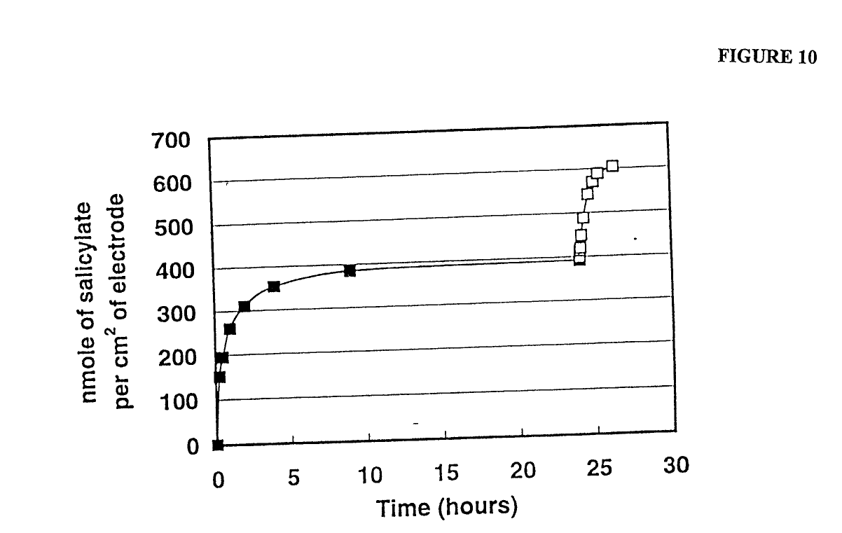

FIGS. 10 and 11 evidence that subjecting a 40% hydrolyzed PVA overlayer to similar crosslinking conditions resulted in a dramatic difference in the salicylate release characteristics. In FIG. 10, the 40% hydrolyzed PVA overlayer was not thermally crosslinked and the results from the release experiments indicate that this system has the same spontaneous release characteristics as the CDD systems with no overlayer. FIG. 11 indicates, that the PP/salicylate system with the crosslinked 40% hydrolyzed PVA overlayer exhibited highly inhibited spontaneous release behavior, with less than 5% of the reservoir being spontaneously released within a 24 hour period of time and more than 350 nmole of salicylate per cm² being released with an applied potential.

### DETAILED DESCRIPTION OF THE INVENTION

The invention herein comprises the use of polypyrrole as the host Ad polymer for a CDD system. The potential for an electrochemically responsive CDD system is extended by including an electro-inactive bilayer.

A major lingering concern regarding a drug delivery system using electroactive polymers has been the spontaneous release of an active molecule(s) by ion exchange. Now, however, the instant invention has eliminated this problem by utilizing a second polymer layer, applied to the top of the electroactive polymer, to represses the undesired spontaneous ion exchange reaction.

Further, in this invention, after applying a coating of 40 mole % hydrolyzed poly(vinyl alcohol) or poly(vinyl butyral) (“PVB”) over a polypyrrole/salicylate (“PPy/Sal”) electrode, the spontaneous release rate is advantageously slowed, while still allowing for a burst release of salicylate by application of a potential to the electroactive polymer.

Polypyrrole based polymer systems of this invention are useful as a CDD system(s) for effective delivery of cationic and anionic biomolecules to humans and animals for medicinal purposes.

FIG. 1 illustrates a simple non-limiting model for mono-anion release from an electroactive polymer film. Illustratively in carrying out this invention, the loading of the anionic drug into the polymer matrix is carried out as part of the polymerization process; whereby the monomer is electropolymerized in the presence of the salt of the anionic dopant. The anionic drug is incorporated into the polymer matrix to maintain charge neutrality of the polymer system. This ion transport into the polymer matrix is driven by electrostatic interactions between the positively charged polymer and the negatively charged dopant ions. Release of the dopant is achieved via reduction of the polymer to its neutral state, causing the dopants to be expelled as charge neutrality is once again maintained. With electrochemically responsive CDD systems there exists the obstacle of spontaneous exchange which causes active molecules to be released when no electrochemical stimulus is applied. To slow this undesired reaction, a polymer bilayer strategy was implemented to help overcome the problem of spontaneous exchange; thereby allowing for a higher degree of control over the release of the biomolecules from the CDD system.

When using polypyrrole as a host-polymer for ionic drug delivery systems, the drug molecule is conveniently incorporated into the polymer matrix as an ionic dopant, not as a covalently bonded moiety. The ionic bond is easier to break than a covalent bond, and provides for a much more efficient system requiring less energy. This allows for a wide variety of drugs and biomolecules to be utilized.

A problem which could occur with an electrochemically responsive drug delivery system is the spontaneous release of drug molecules when no electrochemical stimulus is given. This spontaneous release, usually via ion exchange, of active molecules is not desired since unwanted doses of active molecules, which could be pharmaceutical compounds, could lead to undesired and possibly deleterious interactions.

In order to stop or limit the ion exchange process, a polymer overlayer is deposited on the loaded CDD system to limit the interaction between the active molecules in the CDD system matrix and species of similar charge in the ionic media. Because the CDD system operates in an aqueous ionic media, it is important to have an overlayer which possesses the right combination of hydrophobicity and permeation characteristics.

Drugs useful herein are preferably pharmaceutical compounds selected from the group comprising NSAIDS, analgesics, antihistamines, antitussives, decongestants, expectorants, steroids, enzymes, proteins, antibiotics, hormones, and mixtures thereof and the like.

Nonlimiting examples of such pharmaceutical compounds include but are not limited to nutritional supplements, anti-inflammatory agents (e.g. NSAIDS such as s-ibuprofen, ketoprofen, fenoprofen, indomethacin, meclofentamate, mefenamic acid, naproxen, phenylbutazone, piroxicam, tolmetin, sulindac, and dimethyl sulfoxide), antipyretics, anesthetics including benzocaine, pramoxine, dibucaine, diclonine, lidocaine, mepiracaine, prilocaine, and tetracaine; demulcents; analgesics including opiate analgesics, non-opiate analgesics, non-narcotic analgesics including acetaminophen and astringent including calamine, zinc oxide, tannic acid, Hamamelis water, zinc sulfate; natural or synthetic steroids including triamcinolone, acetonide, prednisone, beclomethasone dipropionate; asthmatic drugs including terbutaline sulfate, albuterol, leukotriene receptor antagonists; electrolytes, metals and minerals; antianxiety and antidepressant agents; antimicrobial and antiviral agents; antihistamines; immune-suppression agents; cholesterol-lowering agents; cardiac and high-blood pressure agents and mixtures thereof and the like.

The examples herein are only illustrations of various embodiments of the instant invention and are not intended to limit the scope of this invention in any way.

### EXAMPLES

1. Loading and Release Studies of PP/Salicylate Systems

The results for each experiment presented is the average of three or more experiments carried out under similar conditions. Polypyrrole films were deposited under an inert atmosphere onto stainless steel electrodes (3 cm²) at constant potential (0.8 V vs. Ag/AgC1) from an aqueous solution of 0.1 M pyrrole and 0.2 M sodium salicylate, with the salicylate acting as both the electrolyte and the dopant. A polymerization time of ca. 15 min. was used to obtain films with a charge density during deposition of 550 mC/cm². Release experiments were carried out in phosphate buffer (ph=7.4) at constant potential (−0.5 V). The amount of salicylate released was determined using UV-Vis spectroscopy.

Poly(vinyl butyral)(PVB) overlayers—PVB overlayers were deposited onto the PP/salicylate films from a 2% PVB/THF solution. The overlayers were allowed to dry at room temperature prior to release studies.

Nafion overlayers—Nafion overlayers were deposited onto the PP/salicylate films from a 5% nafion/alcohol/10% water solution. The overlayers were allowed to dry at room temperature, then heated under vacuum for 1 hour at 150° C. 88 mole % hydrolyzed poly(vinyl alcohol) (PVA) overlayers—88% hydrolyzed PVA overlayers were deposited onto the PP/salicylate films from an aqueous solution containing 5% PVA. The overlayers were allowed to dry at room temperature, then thermally crosslinked under vacuum at 70° C. for 30 min. followed by 30 min. at 150° C. 40 mole % hydrolyzed poly(vinyl alcohol) overlayers—40% hydrolyzed PVA overlayers were deposited onto the PP/salicylate films from an aqueous solution containing 5% PVA. The overlayers were allowed to dry at room temperature, then thermally crosslinked under vacuum at 70° C. for 30 min. followed by 30 min. at 150° C.

FIG. 2 shows that application of any applied potential to reduce the film leads to immediate and rapid salicylate release. In fact, potential dependence on the release was not found as evidenced by the identical release characteristics at 0.0, −0.1, −0.25, and −0.5 V. The term “burst release” has been coined to represent this phenomenon as very little charge is required to trigger essentially a quantitative release of the drug from the electroactive film. It also was observed that immersion of the PP/salicylate in the buffer without any applied potential led to a constant ion release with approximately 33 percent of the electroreleasable drug being spontaneously released over the same time frame as the applied potential release experiments. FIG. 3 shows that under similar spontaneous exchange conditions, the entire drug reservoir can be depleted within a 24 hour period.

The spontaneous release process, represented in FIG. 4, which is encountered in these CDD systems, is believed to be a simple ion-exchange phenomenon in which drug molecules within the polymer matrix are exchanged with species of the same charge existing in the electrolyte media. In fact, it is well known that this ion exchange process is quite facile within such systems. In the case of these experiments, the high ionic strength of the phosphate buffer (20 mM) helps to facilitate the exchange process.

2. Preparation of Overlayers on PP/Salicylate systems and Release Studies

In order to stop or limit this ion exchange process, a strategy was developed whereby a polymer overlayer was deposited on top of the loaded CDD system to limit the interaction between the drug molecules in the CDD system matrix and species of similar charge in the ionic media, as presented in FIG. 5. Because this CDD system operates in an aqueous ionic media, it is important to have an overlayer which possesses the right combination of hydrophobicity and permeation characteristics. The overlayer candidates were then chosen with this in mind.

Poly(vinyl butyral)(PVB) was one of the materials tested as an overlayer. PVB is commonly used as a safety glass interleaver and is well known for its hydrophobic nature. PVB films cast from 0.5, 1, and 2 percent THF solutions and dried at room temperature were used as overlayers for the PP/salicylate system, with the 2 percent solution giving the best results. As shown in FIG. 6, the presence of the PVB overlayer significantly reduced the amount of salicylate spontaneously released over a 24 hour period. The amount spontaneously released went from a quantitative release without the overlayer to approximately ⅓ of the reservoir (100 nmole/cm²) being released when the overlayer was present. Application of an applied potential at the end of this period resulted in the burst release of the drug remaining in the reservoir. This indicates that, although the PVB overlayer does impede spontaneous exchange, it does not change the burst release behavior of the CDD system. FIG. 7 depicts data from an experiment in which the PVB was initially present and then removed using THF after the applied potential release began to subside. Removal of the overlayer allowed for a higher rate of applied potential release to take place. These results indicate that the PVB overlayer not only inhibits spontaneous release, but also hinders normal applied potential release. This data suggests that a truly successful overlayer must exhibit a much higher ratio of applied potential release vs. spontaneous release.

A Nafion film deposited from a 5% solution in 90% alcohol/10% water was also tested as an overlayer. The overlayers were allowed to dry at room temperature, then heated under vacuum for one hour at 150° C. Nafion is a fluoropolymer which is well known for its permselectivity towards cations but not anions. This overlayer was chosen in hopes that it would limit the amount of anions entering the host-polymer matrix. As shown in FIG. 8, salicylate release from the Nafion coated PP/salicylate system exhibited release behavior similar to that of the PVB coated system. However, the ratio of spontaneously release to applied potential release for the Nafion overlayer was approximately 1:3; while the ratio for the PVB overlayer was approximately 1:2. Although this was an improvement over the PVB coated system, the inhibition of spontaneous release is not significant enough to warrant use of Nafion as the preferred overlayer polymer.

Hydrolyzed poly(vinyl acetate)(PVA) derivatives also were tested as overlayer materials. In general, hydrolyzed PVA derivatives are relatively hydrophilic in nature; but can become hydrophobic when undergoing crosslinking. Coatings prepared from 88% hydrolyzed PVA and 40% hydrolyzed PVA were used as overlayers. The 88% hydrolyzed PVA coating was deposited from an aqueous solution containing 5% PVA, while the 40% hydrolyzed PVA coating was deposited from a THF solution containing 5% PVA. The resulting overlayers were then thermally crosslinked in a vacuum oven at 70° C. for 30 minutes then 150° C. for 30 minutes. The PP/salicylate system with the 88% hydrolyzed PVA overlayer was not effective at impeding spontaneous release, as shown in FIG. 9.

It is believed that the 88% hydrolyzed PVA, which contains a high hydroxyl group content and can be viewed as essentially poly(vinyl alcohol), does not undergo thermal crosslinking as well as the 40% hydrolyzed PVA, which contains more acetate groups. This lower degree of crosslinking causes the 88% PVA overlayer to exhibit much poorer spontaneous exchange impedance properties. In fact, there was very little difference observed between the salicylate release behavior of the PP/salicylate system with the 88% hydrolyzed PVA overlayer which was subjected to thermal crosslinking conditions and one without which was not subjected to crosslinking. On the other hand, subjecting the 40% hydrolyzed PVA overlayer to similar crosslinking conditions resulted in a dramatic difference in the salicylate release characteristics, as seen when comparing FIG. 10 to FIG. 11. In FIG. 10, the 40% hydrolyzed PVA overlayer was not thermally crosslinked and the results from the release experiments indicate that this system has the same spontaneous release characteristics as the CDD systems without an overlayer. As FIG. 11 indicates, the PP/salicylate system with the crosslinked 40% hydrolyzed PVA overlayer exhibited highly inhibited spontaneous release behavior, with less than 5% of the reservoir being spontaneously released within a 24 hour period of time and more than 350 nmole of salicylate per cm² being released with an applied potential. This reflects a spontaneous release to applied potential release ratio of 1:19. Compared to the PVB and Nafion overlayers, in which the ratios are 1:2and 1:3respectively, the 40% hydrolyzed PVA overlayer exhibits a much higher degree of spontaneous release impedance. The crosslinking process not only makes the overlayer more hydrophobic, but also more impermeable due to the extended networking within the overlayer matrix. The results from these experiments suggest that in order to successfully impede spontaneous release, the overlayer must not only be made from a hydrophobic material, but also be a highly networked, i.e. crosslinked, material.

One of the key factors in being able to have a high degree of control over the electrochemical release of drugs or biomolecules from a CDD system is the ability to impede spontaneous release. In experiments designed to develop a method to impede this process, it has been found that the use of a hydrophobic, highly networked overlayer is an excellent solution. From the various overlayer materials tested, the crosslinked 40% hydrolyzed PVA gave the best results.

Thus, it is apparent that there has been provided, in accordance with the instant invention, a process that fully satisfies the objects and advantages set forth herein above. While the invention has been described with respect to various specific examples and embodiments thereof, it is understood that the invention is not limited and many alternatives, modifications and variations will be apparent to those n the art in light of the foregoing description. Accordingly, it is intended to all such alternatives, modifications and variations as fall within the spirit and cope of the invention.

What is claimed is:

1. A controlled drug release electrode system comprising an electroactive polymer having an ionic exchangeable releasable dopant thereon and an effective conforming thickness of a water insoluble film forming overlayer substantially impermeable to said dopant.

2. The electrode system of claim 1 wherein said effective conforming thickness is of a thickness sufficient to be substantially impermeable to said dopant.

3. The system of claim 1 wherein said insoluble film forming overlayer comprises a polymer.

4. The system of claim 3 wherein said polymer comprises poly(vinyl butyral), nation, or poly(vinyl acetate).

5. The system of claim 4 wherein the poly(vinyl acetate) is at the most 88% hydrolyzed.

6. The system of claim 4 wherein the poly(vinyl acetate) is less than or equal to 40% hydrolyzed.

7. A process for preparing a controlled drug release electrode system, said system comprising an electroactive polymer having an ionic exchangeable dopant thereon and an effective conforming thickness of a water insoluble film forming overlayer substantially impermeable to said dopant thereon, said process comprising the effective application of said film forming overlayer in an adherent fashion to said polymer.

8. The process of claim 7 wherein said application process is selected from the group consisting of dipping, coating, printing, spraying and vapor deposition of said film forming overlayer to said polymer.

9. The process of claim 7 wherein said film forming overlayer is adherent to said polymer.

10. The process of claim 8 wherein said film forming overlayer comprises a polymer.

11. The process of claim 8 wherein said polymer comprises homopolymers and copolymers of polypyrrole, N-substituted pyrrole and C-substituted pyrrole.

12. The process of claim 11 wherein said polymer comprises polypyrrole.

13. The process of claim 10 wherein said polymer comprises poly(vinyl butyral), nation, or poly(vinyl acetate).

14. A method for treating patients using a controlled drug release electrode system, said system comprising an electroactive polymer having an ionic exchangeable dopant thereon and an effective conforming thickness of a water insoluble film forming overlayer substantially impermeable to said dopant, said method comprising contacting said patient with said electrode system and applying an effective potential to said electrode system whereby said drug is made effectively available to the patient.

15. The method of claim 14 wherein said film forming overlayer comprises a hydrophobic polymer.

16. The method of claim 15 wherein said overlayer is selected from the group consisting of poly(vinyl butyral), poly(vinyl acetate), or Nafion.

17. The method of claim 14 wherein said electroactive polymer comprises homopolymers and copolymers of polypyrrole, N-substituted pyrrole and C-substituted pyrrole.

18. The method of claim 17 wherein said electroactive polymer is polypyrrole.

19. A process of using a polymeric material as a controlled drug delivery system comprising the use of a polymer bilayer containing drug molecules to impede the spontaneous release of said drug molecules when no electrochemical stimulus is applied.

20. The process of claim 19 wherein the polymer bilayer comprises an electroactive polymer and a second polymer layer, where the second polymer layer is applied to the top of the electroactive polymer.

21. The method of claim 20 wherein the second polymer layer is made of hydrophobic material and is crosslinked.

22. The method of claim 20 wherein the second polymer layer is selected from the group consisting of poly(vinyl butyral), Nafion and poly(vinyl acetate).

23. The method of claim 20 and 22 wherein the electroactive polymer is homopolymers and copolymers of polypyrrole, N-substituted pyrrole and C-substituted pyrrole.

24. The method of claim 23 wherein said electroactive polymer is polypyrrole.

25. An article of manufacture comprising a controlled drug release electrode system comprising an electroactive polymer having an ionic exchangeable dopant thereon and additionally an effective conforming thickness of a water insoluble film forming overlayer substantially impermeable to said dopant.

26. The article of manufacture of claim 25 where said article of manufacture is placed in contact with a patient's skin.

27. The article of manufacture of claim 26 wherein an effective potential is applied to said electrode wherein said potential causes the release of said drug, making said drug effectively available to the patient.

28. The method of claim 25 wherein said film forming overlay comprises a polymer made from hydrophobic material which is crosslinked.

29. The method of claim 28 wherein said electroactive polymer comprises homopolymers and copolymers of polypyrrole, N-substituted pyrrole and C-substituted pyrrole.

30. The method of claim 29 wherein said electroactive polymer comprises polypyrrole.

31. A process of creating an electrochemical responsive controlled drug delivery system comprising loading a film of an electroactive polymer with an active ingredient, applying a second polymer layer to said electroactive polymer loaded with an active ingredient, and allowing said second polymer layer to dry.

32. The process of claim 31 where said electroactive polymer comprises homopolymers and copolymers of polypyrrole, N-substituted pyrrole and C-substituted pyrrole.

33. The process of claim 32 wherein said electroactive polymer comprises polypyrrole.

34. The process of claim 33 wherein said electroactive polymer is produced by depositing polypyrrole onto a stainless steel electrode by utilizing a constant potential from an aqueous solution comprising a pyrrole, a salt of an anionic or cationic active ingredient, and a dopant.

35. The process of claims 31 wherein the second polymer layer is poly(vinyl butyral).

36. The process of claim 35 wherein the second polymer layer is allowed to dry at about room temperature.

37. The process of claims 31 wherein the second polymer layer is nation.

38. The process of claim 37 wherein the second polymer layer is allowed to dry at about 150° C. for 1 hour.

39. The process of claim 31 wherein the second polymer layer is poly(vinyl acetate).

40. The process of claim 39 wherein the second polymer layer is allowed to dry at room temperature.

41. The process of claim 42 wherein the second polymer layer is thermally crosslinked in a vacuum for about 30 minutes at 70° C. and then for 30 minutes at 150° C.

42. The process of claim 41 wherein the poly(vinyl acetate) is less than 88% hydrolyzed.

43. The process of claim 42 wherein the poly(vinyl acetate) is about 40% hydrolyzed.

44. A dopant controlled release system comprising an electroactive polymer having an ionic exchangeable releasable dopant thereon and an overlayer to lessen the spontaneous release of said dopant.

45. The system of claim 44 wherein the overlayer is made of a hydrophobic material.

46. The system of claim 45 wherein the overlayer is highly networked.

47. The system of claim 46 wherein the overlayer is highly networked due to crosslinking.

48. The system of claim 45 wherein the overlayer is chosen from the group consisting of poly(vinyl butyral), poly(vinyl acetate), and nation.

49. The system of claim 48 wherein said electroactive polymer comprises homopolymers and copolymers of polypyrrole, N-substituted pyrrole and C-substituted pyrrole.

50. The system of claim 49 wherein said electroactive polymer comprises polypyrrole.

51. The method of claim 45 wherein said dopant is a biologically active ingredient.

52. The method of claim 51 wherein said biologically active ingredient is a pharmaceutical compound.

53. The method of claim 52 wherein said pharmaceutical compound is selected from the group consisting of nutritional supplements, anti-inflammatory agents(e.g. NSAIDS such as s-ibuprofen, ketoprofen, fenoprofen, indomethacin, meclofentamate, mefenamic acid, naproxen, phenylbutazone, piroxicam, tolmetin, sulindac, and dimethyl sulfoxide), antipyretics, anesthetics including benzocaine, pramoxine, dibucaine, diclonine, lidocaine, mepiracaine, prilocaine, and tetracaine; demulcents; analgesics including opiate analgesics, non-opiate analgesics, non-narcotic analgesics including acetaminophen and astringent including calamine, zinc oxide, tannic acid, Hamamelis water, zinc sulfate; natural or synthetic steroids including triamcinolone, acetonide, prednisone, beclomethasone dipropionate; asthmatic drugs including terbutaline sulfate, albuterol, leukotriene receptor antagonists; electrolytes, metals and minerals; antianxiety and antidepressant agents; antimicrobial and antiviral agents; antihistamines; immune-suppression agents; cholesterol-lowering agents; cardiac and high-blood pressure agents and mixtures thereof.
